(19) United States
(12) Patent Application Publication
(10) Pub. No.: US 2005/0197560 A1
(43) Pub. Date: Sep. 8, 2005

(54) System for detecting symptoms, determining staging and gauging drug efficacy in cases of Alzheimer's disease

(76) Inventors: Stephen M. Rao, Shorewood, WI (US); Catherine L. Elsinger, Wauwatosa, WI (US)

Correspondence Address: John Horn;Neurognostics c/o Techstar, Suite 250, 756 N. Milwaukee St., Milwaukee, WI, 53202, US

(21) Appl. No.: 10/793,994

(22) Filed: Mar. 5, 2004

(51) Int. Cl. A61B 5/05

(52) U.S. Cl. 600/410

(57) ABSTRACT

A system for using functional magnetic resonance imaging (fMRI) for detecting symptoms indicative of Alzheimer's disease, diagnosing Alzheimer's disease and gauging the efficacy of medications used in treating Alzheimer's disease. The system includes steps involving activating a selected region of the brain which may be affected by Alzheimer's disease using an identity recognition type task, concurrently acquiring task-active MRI data responsive to the task, comparing the patient's task-active MRI data to reference data derived from a database of task-active data from healthy individuals and detecting whether the patient has symptoms related to Alzheimer's disease. The severity of the patient's symptoms and the staging of the disease may also be determined. Also, a medication may be administered to the patient and the efficacy of the medication may be gauged based on the severity of the patient's symptoms.

### FIELD OF THE INVENTION

The present invention relates to systems for use in detecting symptoms of neurodegenerative disorders and more specifically to using functional magnetic resonance imaging (fMRI) for detecting symptoms, staging and gauging drug efficacy in cases of Alzheimer's disease.

### BACKGROUND OF THE INVENTION

Alzheimer's Disease (AD) is the most common cause of dementia and is a progressive neurodegenerative disorder resulting in gradual deterioration in cognition, function, and behavior. Approximately 2-4 million individuals in the US and more than 30 million worldwide are affected. Age is an important risk factor with AD occurring in 8% of individuals over 65 and 30% over age 85. The progression of AD is gradual with the average patient living 8-10 years after symptom onset. The prevalence of AD is expected to triple over the next 50 years in developed countries. The annual cost of the disease in the United States alone is estimated to be $100 billion. Pathologically, AD is characterized by the appearance of senile (amyloid) plaques and neurofibrillary tangles and by a loss of large cortical neurons in the hippocampus, entorhinal cortex, and association areas of the neocortex. A definitive diagnosis of AD can not be made during life; instead, patients are often provided a provisional diagnosis of possible or probable AD based on clinical, laboratory, and later stage neuroimaging data.

There is increasing evidence that the pathological process associated with AD may begin decades prior to diagnosis. The preclinical stage of AD may be divided into two periods: a “latent” phase with no observable symptoms and a “prodromal” phase characterized by mild symptoms that do not meet diagnostic criteria for probable or possible AD. Early detection of neurodegenerative disorders would enable more effective diagnosis and treatment of AD patients. Preventive therapy, such as anti-amyloid medications, could be usefully started during the preclinical period prior to symptom onset. A delay in onset can result in a 50% decrease in prevalence and a delay of 10 years would result in a disappearance of the disease. Early identification of AD is essential for evaluating and implementing therapies designed to prevent or delay the devastating changes in cognition, behavior, and daily living activities. Presently, identification of “at-risk” individuals typically relies on age, family history, clinical testing, laboratory tests, and genetic screening that are laborious, expensive and unreliable. Approximately 60% of individuals with mild cognitive impairment (MCI), characterized by isolated memory dysfunction, eventually develop AD. However, it is currently not possible to discriminate which MCI subjects will develop a progressive dementia from those who will not.

Positron emission tomography (PET) can provide neuroimaging capabilities useful in the detection of neurodegenerative disorders, and PET resting glucose metabolic studies have demonstrated some promise in the early detection of AD. However, PET has limited spatial and temporal resolution and relies on measuring global indices of resting brain activity which are not specific to the brain systems (e.g., memory) most vulnerable to disruption at the earliest stages of neurodegeneration. PET requires the injection of radioisotopes. This presents safety limitations in the number of studies that can be administered to a given patient over a short period of a time, thereby limiting its ability to monitor drug efficacy. PET also requires the on-site or nearby installation and maintenance of a cyclotron (due to the short half life of radioisotopes used to measure cerebral blood flow), thus generally limiting the installed base of available machines to a small number of academic medical centers.

In U.S. Pat. No. 6,490,472 to Li et al a method is described for producing an indication of the presence of Alhzeimer's disease using a magnetic resonance imaging (MRI) machine to measure functional connectivity. Functional activity and associated connectivity within the hippocampus of a patient's brain is measured while the brain is substantially at rest. The method includes acquiring a series of functional magnetic resonance image (fMRI) data arrays over a period of time to form a time course MRI data set that comprises a set of time domain voxel vectors in which each vector indicates an MRI signal from a different location in the patient's brain. A series of vectors from locations in the brain commonly affected by Alzheimer's disease are then selected and a connectivity index is produced by cross-correlating these vectors. The magnitude of this connectivity index is proposed to be an indicator of the presence of Alzheimer's disease and a quantitative measure of the disease's progress.

fMRI is a neuroimaging technology which has been used in researching functional aspects of central nervous system disorders. fMRI is an application of nuclear or MRI technology in which functional brain activity is detected usually in response to an activation task performed by a patient. fMRI is capable of detecting localized event-related brain activity and changes in this activity over time. Its principal advantages are its strong spatial and temporal resolution and, as no isotopes are used, a virtually unlimited number of scanning sessions that can be performed on a given subject, making within subject designs feasible. fMRI operates by detecting increases in cerebral blood volume that occur locally in association with increased neuronal activity. A widely used fMRI method for detecting brain activity is based upon the blood oxygenation level dependent (BOLD) response. The BOLD signal arises as a consequence of a ‘paradoxical’ increase in blood oxygenation, presumably due to increased local blood flow in excess of local metabolic demand and oxygen consumption following neuronal activity. An increase in blood oxygenation results in increased field homogeneity (increase in T2 and T2*), less dephasing of spins, and increased MR signal on susceptibility-weighted MRI images.

### SUMMARY OF THE INVENTION

It is another object of the present invention to provide a system for gauging the efficacy of drugs in treating Alzheimer's disease using fMRI technology.

It is The present invention comprises a system for detecting symptoms related to Alzheimer's disease, diagnosing and monitoring the progression of the disease and assessing the efficacy of medications in treating the disease. The system uses an MRI scanner to implement a functional magnetic resonance imaging (fMRI) scanning process in which an identity recognition activation task is performed by the patient during an MRI scan. The MRI scanner generates a time image series of MRI scan data showing functional activity in the brain generated by the identity recognition task.

The identity recognition task is employed in order to engage processes related to remote semantic retrieval and stimulate activity in regions of the brain such as the medial temporal and frontal-temporal regions directly affected by Alzheimer's disease. In the preferred embodiment the identity recognition task involves recognizing faces of famous individuals, although other famous images or icons such as famous landmarks, automobiles and even famous names could also be used. The actual activation task encompasses both the recognition of famous faces and the presentation of faces previously not encountered. Identity recognition related MRI data indicative of the functional MRI brain activity of the patient responsive to the task is acquired and recorded. The identity recognition related MRI data are analyzed by making comparisons between these data for the individual patient and standards for functional brain activity responsive to identity recognition tasks derived from reference data from healthy patients. On the basis of these comparisons symptoms related to Alzheimer's disease may be detected and the presence and progress of Alzheimer's disease in the patient may be diagnosed.

In a further embodiment a medication intended to address symptoms related to Alzheimer's disease is administered to the patient. The resulting task-active MRI data from the patient are analyzed and compared with identity recognition task-activated data elicited from the patient when not on medication. The patient's data may also be compared with reference data derived from a reference database including identity recognition activity MRI data from healthy subjects and from subjects known to be afflicted with Alzheimer's disease. The effectiveness of the medication can then be evaluated based on the relative severity of the symptoms detected in said patient.

It is an object of the present invention to provide a system for detecting the symptoms of Alzheimer's disease at an early stage in the development of the disorder using fMRI technology.

It is a further object of the present invention to provide a system for accurately diagnosing Alzheimer's disease and assessing the staging of the disease using fMRI technology.

a yet further object of the present invention to provide a system for detecting the early symptoms of Alzheimer's disease in an efficient, consistent and reliable manner.

It is yet another object of the present invention to provide an activation task for use in fMRI studies for stimulating brain activity in regions of the brain known to be affected by Alzheimer's disease.

### BRIEF DESCRIPTION OF THE DRAWINGS

FIG. 1 provides a diagrammatic illustration of a magnetic resonance imaging machine and its major components as adapted for performing functional magnetic resonance imaging studies.

FIG. 2 provides a flowchart illustrating the operative process for detecting the symptoms, diagnosing and determining the staging of Alzheimer's disease in accordance with the present invention.

FIG. 3 provides a flowchart illustrating the operative process for detecting the symptoms and gauging the efficacy of medications intended to treat Alzheimer's disease in accordance with the present invention.

### DETAILED DESCRIPTION OF THE INVENTION

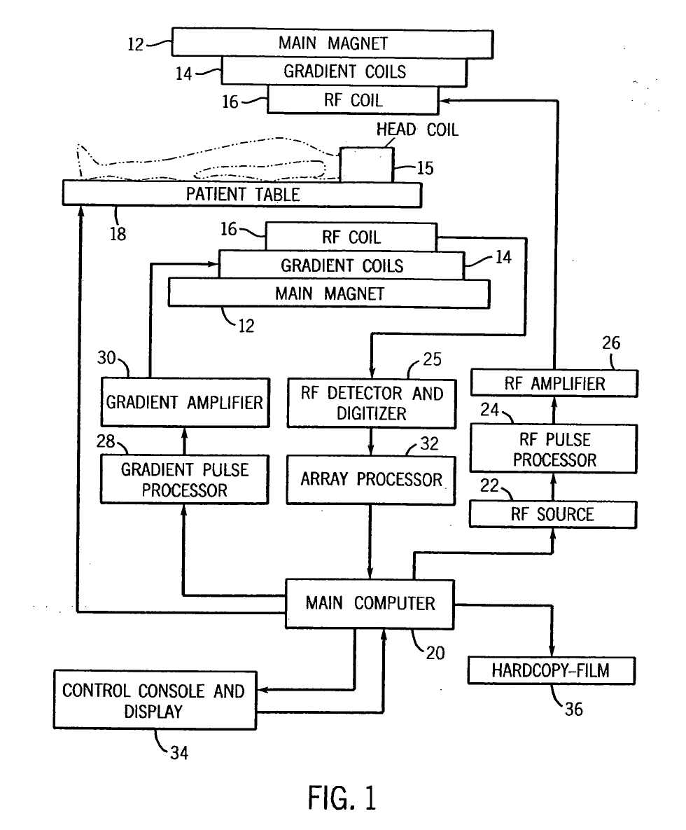

Referring now to FIG. 1, the basic components of a magnetic resonance imaging (MRI) machine 10 are shown. The main magnet 12 produces a strong B. field for the imaging procedure. Within the magnet 12 are the gradient coils 14 for producing a gradient in the Bₒ field in the X, Y, and Z directions as necessary to provide frequency discrimination. A head coil 15 is also used to improve accuracy and resolution for studies involving the brain. Within the gradient coils 14 is a radio frequency (RF) coil 16 for producing RF pulses and the B₁ transverse magnetic field necessary to rotate magnetic spins by 90° or 180°. The RF coil 16 also detects the return signals from the spins within the body and supplies these signals to the RF detector and digitizer 25. The patient is positioned within the main magnet by a computer controlled patient table 18. The scan room is surrounded by an RF shield, which prevents the high power RF pulses from radiating out through the hospital and prevents the various RF signals from television and radio stations from being detected by the imager. The heart of the imager is the computer 20 that controls the components of the imaging system. The RF components under control of the computer include the radio frequency source 22 and pulse programmer 24. The source 22 produces a sine wave of the desired frequency. The pulse programmer 24 shapes the RF pulses into apodized sinc pulses. The RF amplifier 26 greatly increases the power of the RF pulses. The computer 20 also controls the gradient pulse programmer 28 which sets the shape and amplitude of each of the three gradient fields. The gradient amplifier 30 increases the power of the gradient pulses to a level sufficient to drive the gradient coils 14. In most systems an array processor 32 is also provided for rapidly performing two-dimensional Fourier transforms. The computer 20 off-loads Fourier transform tasks to this faster processing device. The operator of the imaging machine 10 provides input to the computer 20 through a control console 34. An imaging sequence is selected and customized by the operator from the console 34. The operator can see the MRI images on a video display located on the console 34 or can make hard copies of the images on a film printer 36.

A General Electric Signa EXCITE 3.0 Tesla MRI scanner is preferably used for implementing the present invention although any of a number of commercial MRI scanners having 3.0 or 1.5 (or less) Tesla fields could be used. General imaging parameters involve, for example, the acquisition of contiguous sagittal slices that cover the entire brain (typically 4 mm thick) using a blipped gradient-echo, echoplanar pulse sequence (echo time (TE)=40 msec; interscan period (TR)=2000 msec; field of view (FOV)=24 cm; 64×64 matrix; 3.75 mL×3.75 mm in-plane resolution). High resolution (124 axial slices) spoiled GRASS (gradient-recalled at steady-state) sagittal anatomic images [TE=5 ms; TR (repetition time)=24 ms, 40° flip angle, NEX (number of excitations)=2, slice thickness=1.5 mm, FOV=24 cm, slice plane=coronal, matrix size=256×128] are acquired prior to functional imaging for anatomical localization of functional activation (duration 20 min.). In addition, Proton Density (PD) and T2-weighted images [TE=36 msec (for PD) or 96 msec (for T2), TR=3000 msec, NEX=1, FOV=26, slice thickness=3.0 mm, slice plane=coronal, matrix=256×192, and an echo train length=8] are acquired simultaneously over seven minutes. The use of three different pulse sequences may facilitate classification of tissue type in the images using discriminate analysis techniques. Stimulus presentation and general communication to the patient in the MR scanner is accomplished with stereo audio headphones and computer generated images fed into a digital LCD projector which are back projected to the subject and viewed by the patient through prismatic glasses. Subject responses are recorded on a small hand held keyboard including multiple buttons. Response data, including task responses, accuracy, RT and choice selection, are acquired on a PC for off-line analysis.

Foam padding is preferably used to limit head motion within the head coil. Head movement, typically subvoxel (<2 mm), is viewed in cine format. The image time series is spatially registered to minimize the effects of head motion and a 3D volume registration algorithm is used align each volume in each time series to a fiducial volume through a gradient descent in a nonlinear least squares estimation of six movement parameters (3 shifts, 3 angles).

The identity recognition activation task includes making familiarity judgments of famous and unfamiliar faces while undergoing fMRI scanning. The stimuli comprise famous faces of well-known entertainers, politicians, criminals, and sports figures. The unfamiliar faces are matched to the famous faces on the basis of demographics (age, gender) and stylistic qualities (e.g., glamour poses). The stimuli consist of grayscale images with background and clothing removed and replaced by a uniform gray color. The pictures were selected to avoid strong facial expressions (e.g., laughter, scowl). The famous faces are tested with a random group of adults to verify that they should be recognized by at least 90% of the participants.

Identity recognition tasks involving semantic memory retrieval activate a common set of brain regions, including the medial and anterolateral temporal regions and posterior cingulate. These regions are typically the first sites of pathological involvement in patients with AD. Further, older healthy subjects show more, rather than less, brain activity in regions associated with person identification tasks relative to young subjects, suggesting that BOLD-based fMRI is a robust measure for measuring brain activity in older adults. Also, famous faces provide an effective stimulus format in older subjects since they tend to generate greater attention and interest in older adults.

The unique activation patterns associated with fMRI identity recognition tasks involving semantic memory retrieval provide an effective marker of cognitive decline in early AD and cognitive decline in mild cognitive impairment (MCI) which is likely to be associated with AD. Declines in episodic memory (memory for information placed in a distinct spatial-temporal context) is associated with both AD and healthy aging (although to significantly different degrees), whereas semantic memory (knowledge of facts about the world that are not tied to a distinct spatial-temporal context) is typically preserved during healthy aging but is impaired in dementia, with such impairments tending to indicate AD and track the clinical course of the disease. Accordingly, fMRI measures of semantic memory performance (and alterations in the neural substrates subserving semantic memory) responsive to identity recognition tasks and can enable the early detection of AD and tracking of the course of the disorder.

During the activation task famous faces, unfamiliar faces, and baseline trials (fixation to a central cross image) are presented randomly in each imaging run. Each stimulus remains on the screen for the duration of each trial which lasts 6 seconds. In all, 80 faces are presented including 40 familiar faces, 40 unfamiliar faces as well as 40 baseline trials (inactive periods). The 120 trials are presented randomly over 2 imaging runs with each run extending 6 minutes in duration. Practice trials are administered and monitored for accuracy to ensure that the subject is fully responsive and understands the task demands. Participants indicate if they recognize the stimuli by pressing one of two keys with the right index or right middle finger. Participants press the left key if the face is famous and the right key if the face is unfamiliar.

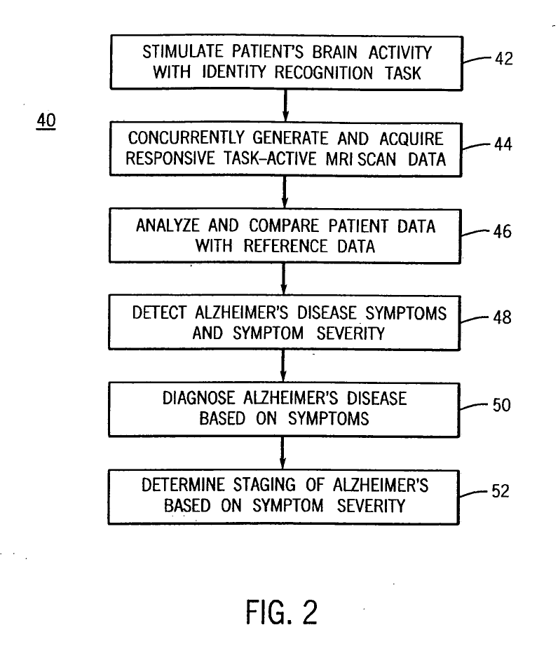

Referring now to FIG. 2, the operative process 40 for detecting the symptoms, diagnosing and determining the staging of Alzheimer's disease includes the steps 42, 44, 46, 48, 50 and 52. In step 42 the patient is stimulated using an identity recognition task in order to generate activity in regions of the patient's brain that may be affected by Alzheimer's disease. An image of a famous person is visually presented to the patient and the patient is required to recognize whether this person is famous and respond accordingly. Alternatively, patients may be presented with the images of famous landmarks or simply the names or titles of famous persons or landmarks. Step 44 is performed concurrently with step 42 so that scanning and data acquisition take place by the MRI machine as brain activity is activated in response to the identity recognition task. In step 44 identity recognition related MRI data indicative of the functional MRI brain activity of the patient responsive to the identity recognition task is acquired and recorded by the MRI scanning system. The identity recognition related MRI data is then analyzed in step 46 by making comparisons between the patient's identity recognition related data, or indexes derived from these data, and reference data, indexes, or standards for functional brain activity responsive to identity recognition tasks derived from MRI data from healthy subjects and from patients known to be afflicted with Alzheimer's disease. In step 48 the presence and severity or the absence of one or more symptoms related to Alzheimer's disease are detected based on these comparisons. Accordingly, in step 50 the patient is diagnosed as having or not having the disease based on the symptoms detected. If the patient is in fact diagnosed with the disease the staging (state of progression) of Alzheimer's disease is determined in step 52 based on the severity of said symptoms detected in the patient.

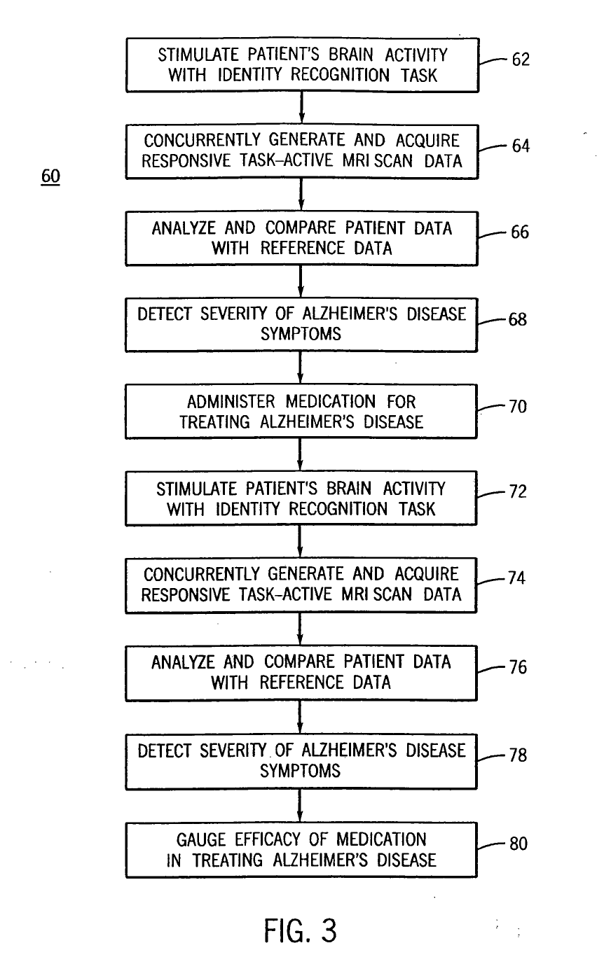

Referring now to FIG. 3, the operative process 60 for detecting the symptoms and gauging the efficacy of medications intended to treat Alzheimer's disease includes the steps 62, 64, 66, 68, 70, 72, 74, 76, 78 and 80. Steps 62, 64, 66 and 68 are similar to steps 42, 44, 46 and 48 as described above and involve activating a selected region of the brain using an identity recognition type task, concurrently acquiring task-active MRI data responsive to the identity recognition task, comparing the patient's MRI data to reference data from healthy individuals and detecting the relative severity of the symptoms of Alzheimer's disease in the patient. In step 70 a medication intended to treat Alzheimer's disease is administered to the patient. Steps 72, 74, 76, and 78 are again similar to steps 42, 44, 46 and 48 as described above and involve activating a selected region of the brain using an identity recognition type task, concurrently acquiring task-active MRI data responsive to the identity recognition task, comparing the patient's MRI data to reference data from healthy individuals and detecting the relative severity of the symptoms of Alzheimer's disease in the patient. However, in step 80 the effectiveness of the medication administered in step 70 is gauged based on the relative severity of the symptoms detected in the patient when under the medication and when not under the medication.

The imaging analysis consists of a comparison of the intensity and extent of regional cerebral activity with respect to famous and unfamiliar faces arising with respect to the activation task. Region of Interest (ROI) analyses are focused on the temporal lobe (specifically the medial and anterolateral regions), the hippocampus and the posterior cingulate. Only correct trials (recognition of targets; rejection of foils) enter into the analyses. Correct trials are verified by a post-scanning questionnaire to determine if in fact the participant was familiar with the famous persons.

Several publicly available software programs such as AFNI (Medical College of Wisconsin in Milwaukee, Wis.) and BrainVoyager (Brain Innovation B.V. in Maastricht, Netherlands) have been developed that allow for whole-brain, 3D fMRI activation mapping and within- and between-subjects statistical comparisons and also include extensive statistical routines. Typically, all whole-brain fMRI data are converted to 4D data sets (time plus 3 spatial dimensions). Functional images are directly registered upon high resolution anatomical scans obtained in the same imaging session. Location and intensity of activation from individual or grouped data are translated into 3D proportionally measured, stereotaxic coordinates relative to the line between the anterior and posterior commissures.

Functional images are first time-locked to the events of interest (e.g., correct recognition of a famous face) and typically averaged to obtain a mean signal response for each voxel. This procedure requires long interstimulus intervals (ISI>14 sec.) to allow the hemodynamic response to return to baseline. Alternatively, a deconvolution analysis program may be used to extract the hemodynamic response (impulse response function-IRF) for each type of stimuli from the time series. A software program such as 3dDeconvolve (AFNI) can estimate the system IRF and can do so even in cases where the ISI is substantially shorter than the hemodynamic response (4 second) which can be a significant analytic advantage for many experimental designs. This program uses a sum of scaled and time-delayed versions of the stimulus time series, with the data itself determining (within limits) the functional form of the estimated response. The program yields the best linear least-squares fit for the following model parameters: constant baseline, linear trend in time series, and estimates the IRF for 7-9 images post-stimulus onset (14-18 sec.) for each condition relative to a baseline state. In a typical imaging run, approximately 33% of “trials” involve a baseline control condition to introduce “jitter” in the time series. Active trials are coded by condition (e.g., famous, unfamiliar) and accuracy (correct, incorrect).

High resolution anatomical and functional images are linearly interpolated to volumes with 1 mm³ voxels, co-registered, and converted to stereotaxic coordinate space. Functional images are typically blurred using a 4 mm Gaussian full-width half-maximum (FWHI) filter to compensate for intersubject variability in anatomic and functional anatomy. Voxel-wise statistical analyses across fMRI 3-D data sets are achieved with 3dANOVA type models (applicable to both within- and between-subject designs). Instead of using the individual voxel probability threshold alone, probability thresholding is used in combination with minimum cluster size thresholding. The underlying principle is that true regions of activation will tend to occur over contiguous voxels, whereas noise has much less of a tendency to form clusters of activated voxels. By combining the two, the power of the statistical test is greatly enhanced. If desired the tradeoff between probability and cluster threshold can be adjusted to achieve the desired significance level. By iteration of the process of random image generation, Gaussian filtering (to simulate spatial correlation between voxels), thresholding, and tabulation of cluster size frequencies, a Monte Carlo simulation program such as AphaSim can be used to generate an estimate of the overall significance level achieved for various combinations of individual voxel probability threshold and cluster size threshold, assuming spatially uncorrelated voxels.

While voxel-wise statistical analyses are easy to implement, they may distort information due to normal variations in cortical and subcortical topography. These differences become magnified when comparing brain activation patterns across groups of subjects (healthy vs. MCI vs. mild AD). In the preferred embodiment information is combined from the SPGR, PD and T2 MR scans and tissue typing (gray matter, white matter, CSF) analyses used to generate measures of atrophy. In addition, there are several regions and subregions of the brain that comprise specific regions of interest (ROIs) to be analyzed in greater detail. The frontal, temporal, and hippocampal regions are parcellated and the posterior cingulate region subdivided to form ROIs. As a part of the overall analyses three dependent values are calculated for each such region of interest (ROI): (1) the number of activated voxels divided by the total number of voxels in the region, a measure of the spatial extent of the activated region, (2) the mean % area-under-the-curve (% AUC) of the activated voxels, a measure of the intensity of the activated region, and (3) a power function defined as the percent of activated voxels in an ROI multiplied by the mean % AUC, an index that combines spread and intensity information.

Although the invention has been described with reference to certain embodiments for which many implementation details have been described, it should be recognized that there are other embodiments within the spirit and scope of the claims and the invention is not intended to be limited by the details described with respect to the embodiments specifically disclosed. For example, semantic retrieval activity may be invoked by other the identity recognition activation tasks such as tasks involving the recognition of famous landmarks, automobiles and names.

1. In a functional MRI scanning process in which an activation task is performed during an MRI scan for the purpose of generating functional activity data, the process including the steps comprising:
a) stimulating a patient using an identity recognition task in order to activate regions of the brain known to be affected by Alzheimer's disease;
b) acquiring and recording a first set of identity recognition related MRI data indicative of the functional MRI brain activity of the patient responsive to said identity recognition task;
c) analyzing said identity recognition related MRI data by making comparisons between said first identity recognition related data of said patient and standards for functional brain activity responsive to identity recognition tasks derived from MRI data from healthy patients;
d) detecting one or more symptoms related to Alzheimer's disease in said patient based on said comparisons.

2. The process of claim 1, wherein:
said step of stimulating a patient includes the steps of visually presenting an image of a famous person to a patient and having said patient recognize whether said person is famous.

3. The process of claim 1, wherein:
said regions of the brain known to be affected by Alzheimer's disease include the temporal lobe, hippocampus and the posterior cingulate.

4. The process of claim 1, further including the step of:
diagnosing Alzheimer's disease based on said symptoms detected in the patient.

5. The process of claim 1, further including the steps of:
analyzing said identity recognition related MRI data by also making comparisons between said data of said patient and standards for identity recognition functional brain activity derived from identity recognition MRI data associated with patients known to be afflicted with Alzheimer's disease; and
detecting the severity of one or more symptoms related to Alzheimer's disease in said patient based on said comparisons.

6. The process of claim 5, further including the step of:
determining the staging of the Alzheimer's disease based on the severity of said symptoms detected in said patient.

7. The process of claim 1, further including the steps of:
administering a medication to said patient intended to address symptoms related to Alzheimer's disease;
stimulating said patient using said identity recognition task in order to activate said regions of the brain known to be affected by Alzheimer's disease while said patient is under medication;
acquiring and recording identity recognition related MRI data indicative of the functional MRI brain activity of the patient responsive to said identity recognition task;
comparing the identity recognition related MRI data acquired while said patient is off medication with said identity recognition related MRI data acquired while said patient is on medication; and
gauging the effectiveness of said medication based on the results of comparing said data.

8. In a functional MRI scanning process in which an activation task is performed during an MRI scan for the purpose of generating functional activity data, the process including the steps comprising:
a) stimulating a patient using an identity recognition task in order to activate regions of the brain known to be affected by Alzheimer's disease;
b) acquiring and recording identity recognition related MRI data indicative of the functional MRI brain activity of the patient responsive to said identity recognition task;
c) analyzing said identity recognition related MRI data by making comparisons between said data of said patient and standards for identity recognition functional brain activity derived from identity recognition MRI data associated with healthy patients and patients known to be afflicted with Alzheimer's disease; and
d) detecting the severity of one or more symptoms related to Alzheimer's disease in said patient based on said comparisons.

9. The process of claim 8, further including the step of:
determining the staging of the Alzheimer's disease based on the severity of said symptoms detected in the patient.

10. The process of claim 8, further including the steps of:
administering a medication to said patient intended to address symptoms related to Alzheimer's disease as the first step in said process, and
gauging the effectiveness of said medication based on the severity of the symptoms detected in said patient.

11. The process of claim 8, wherein:
said step of stimulating a patient includes the steps of visually presenting an image of a famous person to a patient and having said patient recognize whether said person is famous.

12. The process of claim 8, wherein:
said step of stimulating a patient includes the steps of visually presenting an image of a famous landmark to a patient and having said patient recognize whether said landmark is famous.

13. The process of claim 8, further including the step of:
diagnosing Alzheimer's disease based on the severity of said symptoms detected in said patient.

14. A system for detecting functional symptoms related to Alzheimer's disease using an MRI scanner, comprising the steps of:
a) activating a selected region of the brain known to be affected by Alzheimer's disease by having a patient perform an identity recognition task;
b) repeatedly acquiring MRI data using an MRI scanner to produce a time image series including task-active MRI data indicative of task-activated brain activity of the patient in the selected region;
c) comparing said task active MRI data from said patient with reference data derived from a reference database including task-active MRI data from healthy subjects for identity recognition task-activated brain activity in the selected region; and
d) detecting one or more symptoms of Alzheimer's disease based on the results of comparing said patient data and reference data.

15. The system of claim 14, wherein:
said step of comparing includes selecting and adapting the reference data from said database for specific application to said patient according to the medical condition of the patient.

16. The system of claim 14, wherein:
said step of activating a selected region includes the step of visually presenting an image of a famous person to a patient.

17. The system of claim 14, wherein:
said step of activating a selected region includes the step of visually presenting an image of a famous landmark to a patient.

18. The system of claim 14, further including the step of:
diagnosing Alzheimer's disease based on said symptoms detected in said patient.

19. The system of claim 14, wherein:
said step of comparing also includes comparing said task active MRI data from said patient with reference data derived from a reference database including task-active MRI data from subjects known to be afflicted with Alzheimer's disease for identity recognition task-activated brain activity in the selected region, and
said step of detecting symptoms includes detecting the relative severity of said symptoms in said patient.

20. The system of claim 19, further including the step of:
determining the staging of the Alzheimer's disease based on the severity of said symptoms detected in the patient.

21. The system of claim 19, further including the steps of:
administering a medication to said patient intended to address symptoms related to Alzheimer's disease, and
gauging the effectiveness of said medication based on the relative severity of the symptoms detected in said patient.

22. A system for detecting functional symptoms related to Alzheimer's disease using an MRI scanner, comprising the steps of:
a) activating a selected region of the brain known to be affected by Alzheimer's disease by having a patient perform an identity recognition task;
b) repeatedly acquiring MRI data using an MRI scanner to produce a time image series including first task-active data indicative of task-activated brain activity of the patient in the selected region;
c) comparing said first task-active data from said patient with reference data derived from a reference database including task-activated data from healthy subjects and from subjects known to be afflicted with Alzheimer's disease for identity recognition task-activated brain activity in the selected region;
d) detecting the relative severity of one or more symptoms of Alzheimer's disease based on the results of comparing said first patient data and reference data;
e) administering a medication to said patient for the purpose of addressing symptoms related to Alzheimer's disease;
f) activating a selected region of the brain known to be affected by Alzheimer's disease by having said patient perform an identity recognition task;
g) repeatedly acquiring MRI data using an MRI scanner to produce a time image series including second task-active data indicative of task-activated brain activity of the patient in the selected region when under said medication;
h) comparing said second task-active data from said patient with reference data derived from a reference database including task-activated data from healthy subjects and from subjects known to be afflicted with Alzheimer's disease for identity recognition task-activated brain activity in the selected region;
i) detecting the relative severity of one or more symptoms of Alzheimer's disease based on the results of comparing said second patient data and reference data; and
j) gauging the effectiveness of said medication based on the relative severity of the symptoms detected in the patient when under said medication and when not under said medication.

23. The system of claim 22, wherein:
said step of comparing includes selecting and adapting said reference data from said database for specific application to said patient according to the medical condition of the patient.

24. The system of claim 22, wherein:
said step of activating a selected region includes the step of visually presenting an image of a famous person to a patient.

25. The system of claim 22, wherein:
said step of activating a selected region includes the step of visually presenting an image of a famous landmark to a patient.

26. A system for assessing functional symptoms related to Alzheimer's disease using and MRI scanner, comprising the steps of:
a) activating a selected region of the brain in a patient by having the patient perform an identity recognition task while in an MRI scanner;
b) acquiring brain activity MRI data responsive to said task for said selected region in said patient using the MRI scanner;
c) generating a patient index of task active central nervous system activity in said selected region for said patient from said MRI data;
d) comparing said index for said patient with a reference index of task active central nervous system activity derived from database data from healthy individuals for central nervous system activity responsive to said identity recognition task;
e) detecting symptoms of Alzheimer's based on the results of comparing said patient and reference indices.

27. The system of claim 26, wherein:
said step of activating a region includes the steps of visually presenting an image of a famous person to the patient.

28. The system of claim 26, wherein:
said step of activating a region includes the steps of visually presenting an image of a famous landmark to a patient.

29. The system of claim 26, further including the step of:
diagnosing Alzheimer's disease based on said symptoms detected in the patient.

30. A system for assessing the functional efficacy of medications for Alzheimer's disease using and MRI scanner, comprising the steps of:
a) activating a selected region of the brain by having a patient perform an identity recognition task while in an MRI scanner;
b) acquiring a first set of brain activity data responsive to said task for said selected region in said patient using the MRI scanner;
c) generating a first index of task active central nervous system activity in said selected region for said patient from said first set of data;
e) administering a medication to said patient for the purpose of addressing symptoms related to Alzheimer's disease;
d) activating said selected region of the brain by having a patient perform an identity recognition task while in an MRI scanner;
e) acquiring a second set of brain activity data responsive to said task for said selected region in said patient using the MRI scanner;
f) generating a second index of task active central nervous system activity in said selected region for said patient while under medication from said second set of data;
g) comparing said indices representing task active brain activity in said selected region while said patient is off and on said medication; and
h) determining the efficacy of said medication based on the results of comparing said indices.

31. The system of claim 30, wherein:
said step of activating includes the steps of visually presenting an image of a famous person to a patient.

32. The system of claim 30, wherein:
said step of activating includes the steps of visually presenting an image of a famous landmark to a patient.
